(19) United States
(12) Patent Application Publication
(10) Pub. No.: US 2025/0169366 A1
(43) Pub. Date: May 22, 2025

(54) ULTRASOUND PROBES AND ULTRASOUND IMAGING DEVICES

(71) Applicant: WUHAN UNITED IMAGING HEALTHCARE CO., LTD., Wuhan (CN)

(72) Inventors: Kang SI, Shanghai (CN); Zhihao ZHU, Wuhan (CN); Lei ZHANG, Wuhan (CN)

(73) Assignee: WUHAN UNITED IMAGING HEALTHCARE CO., LTD., Wuhan (CN)

(21) Appl. No.: 18/949,958

(22) Filed: Nov. 15, 2024

(30) Foreign Application Priority Data
Nov. 20, 2023 (CN) 202311557905.4

(51) Int. Cl. H10N 30/87; B06B 1/06; G01N 29/24

(52) U.S. Cl. CPC H10N 30/875; B06B 1/0622; G01N 29/2437; H10N 30/872

(57) ABSTRACT

Embodiments of the present disclosure provide an ultrasound probe. The ultrasound probe includes a piezoelectric layer, a backing layer, a first connector, and a second connector. The backing layer is provided in a laminated arrangement with the piezoelectric layer. The first connector is disposed between the piezoelectric layer and the backing layer. The first connector is electrically connected to the piezoelectric layer. The first connector is configured as a flexible circuit board. One end of the second connector is connected to the first connector and the other end is connected to a host.

### CROSS-REFERENCE TO RELATED APPLICATIONS

This application claims priority to Chinese Patent Application No. 202311557905.4, filed on Nov. 20, 2023, the contents of which are incorporated herein by reference in their entirety.

### TECHNICAL FIELD

This application relates to the technical field of medical devices, and in particular, to ultrasound probes and ultrasound imaging devices.

### BACKGROUND

The intracardiac ultrasound probe generally includes a piezoelectric layer, an adapter board, a microwave beamforming integrated circuit (IC), an electrical component, a bonding point, an application specific integrated circuit (ASIC), and a coaxial cable. The microwave beamforming IC, the electrical component, the bonding point, etc., are all integrated into the adapter board. The ASIC is provided under the piezoelectric layer, and electrical signals in the piezoelectric layer are led out through the microwave beamforming IC and then transmitted to the adapter board through the bonding point. The adapter board is then connected to a coupler via a catheter through the coaxial cable, thus forming a complete electrical signal conduction path.

However, in the ultrasound probe described above, the adapter board is generally rigid, with the ASIC arranged below the piezoelectric layer and integrated in the front end of the rigid adapter board. In addition, the rear of the rigid adapter board is also integrated with electronic components cooperating with the ASIC and pads soldering to the coaxial cable. This arrangement results in that the length of the rigid adapter board cannot be minimized, as it contributes an additional portion beyond the base of the piezoelectric layer, and the additional portion is a rigid material that cannot be bent. This necessitates a larger space for the front end of the ultrasound probe to bend during use, making it challenging to operate effectively within the narrow confines of the heart chambers, thereby reducing imaging quality. Moreover, the use of the coaxial cable for connection is constrained by the physical dimension of the cable and the limited internal space of the chamber, hindering the ability to support the electrical signal extraction needs of multi-element probes.

Therefore, it is desirable to provide an ultrasound probe and an ultrasound imaging device to solve the problem of the long length of the ultrasound probe and the inability to support the multi-element probes.

### SUMMARY

Embodiments of the present disclosure provide an ultrasound probe. The ultrasound probe may include a piezoelectric layer, a backing layer, a first connector, and a second connector. The backing layer may be provided in a laminated arrangement with the piezoelectric layer. The first connector may be disposed between the piezoelectric layer and the backing layer. The first connector may be electrically connected to the piezoelectric layer. The first connector may be configured as a flexible circuit board. One end of the second connector may be connected to the first connector and the other end is connected to a host.

In some embodiments, the first connector may include a sound head flexible circuit board. The sound head flexible circuit board may include a sound head circuit board body and one or more connecting portions disposed on at least a portion of a periphery of the sound head circuit board body. The sound head circuit board body may be disposed between the piezoelectric layer and the backing layer. The one or more connecting portions may be connected to the second connector.

In some embodiments, the one or more connecting portions may include a first connecting segment. The first connecting segment may be disposed on the periphery of the sound head circuit board body and may bend away from the piezoelectric layer.

In some embodiments, the one or more connecting portions may further include a second connecting segment. the second connecting segment may be connected to the first connecting segment and may extend over a backside of the backing layer away from the piezoelectric layer.

In some embodiments, the one or more connecting portions may further include a third connecting segment provided at one end of the second connecting segment along a length direction of the second connecting segment. The second connector may be connected to the third connecting segment.

In some embodiments, the one or more connecting portions may include a plurality of connecting portions. The sound head circuit board body may be provided with the plurality of connecting portions at at least one end of the sound head circuit board body along a width direction of the sound head circuit board body. The plurality of connecting portions may be sequentially arranged in at least one row, each row of the at least one row being along a length direction of the piezoelectric layer.

In some embodiments, two adjacent connecting portions among the plurality of connecting portions at a same end of the sound head circuit board body along the width direction of the sound head circuit board body may be disconnected.

In some embodiments, the plurality of connecting portions may be provided in pairs. Two connecting portions in a same pair of connecting portions may be disposed at two end of the sound head circuit board body along the width direction of the sound head circuit board body.

In some embodiments, the ultrasound probe may further include an integrated circuit and one or more electronic components. The integrated circuit may be provided between the piezoelectric layer and the sound head flexible circuit board and may be electrically connected to the piezoelectric layer and the sound head flexible circuit board. The integrated circuit may extend outside the piezoelectric layer from one end of the piezoelectric layer along a length direction of the piezoelectric layer. The one or more electronic components may be disposed on an end of the integrated circuit extending outside of the piezoelectric layer.

In some embodiments, the second connector may include at least one catheter flexible circuit board. The at least one catheter flexible circuit board may be electrically connected to the first connector.

In some embodiments, the first connector may include a sound head flexible circuit board. The sound head flexible circuit board may include a sound head circuit board body and one or more connecting portions disposed on at least a portion of a periphery of the sound head circuit board body. The sound head circuit board body may be disposed between the piezoelectric layer and the backing layer. The one or more connecting portions may be connected to the at least one catheter flexible circuit board. For each of the at least one catheter flexible circuit board, the catheter flexible circuit board may include a catheter circuit board body and one or more lead-out segments. The one or more lead-out segments may be located at at least one end of the catheter circuit board body in a width direction of the catheter circuit board body, and the one or more lead-out segments may be electrically connected to the one or more connecting portions.

In some embodiments, the at least one catheter flexible circuit board may include a plurality of catheter flexible circuit boards. Each of a plurality of catheter circuit board bodies of the plurality of catheter flexible circuit boards may be provided with the one or more lead-out segments at two ends of the catheter circuit board body along the width direction of the catheter circuit board body. Two lead-out segments among the one or more lead-out segments on a same catheter flexible circuit board may be connected to a pair of connecting portions among the one or more connecting portions in a one-to-one correspondence, respectively.

In some embodiments, the at least one catheter flexible circuit board may include a plurality of catheter flexible circuit boards. Each of a plurality of catheter circuit board bodies of the plurality of catheter flexible circuit boards may be provided with the one or more lead-out segments at one end of the catheter circuit board body along the width direction of the catheter circuit board body. A pair of connecting portions among the one or more connecting portions may be connected to two lead-out segments respectively disposed on two catheter flexible circuit boards among the plurality of catheter flexible circuit boards.

In some embodiments, the one or more connecting portions may be welded, bonded, bound together or integrally molded with the one or more lead-out segments.

In some embodiments, for each of the at least one catheter flexible circuit board, the catheter flexible circuit board may include a first flexible circuit board and a second flexible circuit board connected to each other. An end of the first flexible circuit board away from the second flexible circuit board may be connected to the first connector. An end of the second flexible circuit board away from the first flexible circuit board may be connected to the host.

In some embodiments, the first flexible circuit board may include a first transmission end. The second flexible circuit board may include a plurality of second transmission ends. The plurality of second transmission ends may be spaced sequentially along an extension direction of the second flexible circuit board. The first transmission end may be selectively connected to one of the plurality of second transmission ends.

In some embodiments, the ultrasound probe may include a first portion and a second portion connected in sequence. The first connector and the first flexible circuit board may be located within the first portion. The second flexible circuit board may be located within the second portion. The second flexible circuit board may be configured in a helical structure.

In some embodiments, the at least one catheter flexible circuit board may include at least one flexible circuit board in a catheter.

Embodiments of the present disclosure provide an ultrasound imaging device including a host, a coupler, and an ultrasound probe connected in sequence. One end of the ultrasound probe may be connected to the host through the coupler. The ultrasound probe may include a piezoelectric layer, a backing layer provided in a laminated arrangement with the piezoelectric layer, a first connector, and a second connector. The first connector may be disposed between the piezoelectric layer and the backing layer. The first connector may be electrically connected to the piezoelectric layer. The first connector may be configured as a flexible circuit board (e.g., as shown by “F1” in FIG. 11 and FIG. 12). One end of the second connector may be connected to the first connector and the other end is connected to a host.

Embodiments of the present disclosure provide an ultrasound probe. The ultrasound probe may include a piezoelectric layer, a backing layer provided in a laminated arrangement with the piezoelectric layer, a first connector electrically connected to the piezoelectric layer, and a second connector. One end of the second connector may be connected to the first connector and the other end is connected to a host. The second connector may be configured as at least one flexible circuit board (e.g., as shown by “F2” in FIGS. 9-12), and the at least one flexible circuit board may be in at least one catheter.

In some embodiments, the ultrasound probe may include a piezoelectric layer, a backing layer, a first connector, and a second connector. The backing layer may be provided in a laminated arrangement with the piezoelectric layer. The first connector may be disposed between the piezoelectric layer and the backing layer. The first connector may be electrically connected to the piezoelectric layer. The first connector may be configured as a flexible circuit board. One end of the second connector may be connected to the first connector and the other end may be connected to a host. Ultrasonic signals of the piezoelectric layer are transmitted to the host through the first connector and the second connector in sequence. The first connector may be configured as a flexible circuit board, which eliminates the need to set up an adapter board, an application specific integrated circuit (ASIC), and electronic components cooperating with the ASIC. In this way, the ultrasound probe may have a simple structure, which reduces the count of components at a front end of the ultrasound probe, shortens a length of a rigid portion of the ultrasound probe, reduces a volume of a sound head portion, and improves the maneuverability of the ultrasound probe.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a schematic diagram illustrating an internal structure of an exemplary ultrasound probe according to some embodiments of the present disclosure;

FIG. 2 is a schematic diagram illustrating an exemplary sound head flexible circuit board according to some embodiments of the present disclosure;

FIG. 3 is a schematic diagram illustrating an exemplary sound head flexible circuit board according to some other embodiments of the present disclosure;

FIG. 4 is a schematic diagram illustrating an internal disassembled structure of an exemplary ultrasound probe according to some other embodiments of the present disclosure;

FIG. 5 is a schematic diagram illustrating an internal disassembled structure of an exemplary ultrasound probe according to some other embodiments of the present disclosure;

FIG. 6 is a left view of the ultrasound probe shown in FIG. 5 after assembly;

FIG. 7 is a schematic diagram illustrating an exemplary sound head flexible circuit board according to some other embodiments of the present disclosure;

FIG. 8A is a sectional view in a width direction of an exemplary ultrasound probe according to some other embodiments of the present disclosure;

FIG. 8B is a sectional view in a width direction of an exemplary ultrasound probe according to some other embodiments of the present disclosure;

FIG. 8C is a sectional view in a width direction of an exemplary ultrasound probe according to some other embodiments of the present disclosure;

FIG. 9 is a schematic diagram illustrating an exemplary catheter flexible circuit board according to some embodiments of the present disclosure;

FIG. 10 is a schematic diagram illustrating an exemplary catheter flexible circuit board according to some other embodiments of the present disclosure;

FIG. 11 is a schematic diagram illustrating a structure in which an exemplary sound head flexible circuit board is connected to an exemplary catheter flexible circuit board according to some embodiments of the present disclosure;

FIG. 12 is a schematic diagram illustrating a structure in which an exemplary sound head flexible circuit board is connected to an exemplary catheter flexible circuit board catheter flexible circuit board according to another embodiment of the present disclosure;

FIG. 13 is a schematic diagram illustrating structures of an exemplary first flexible circuit board and an exemplary second flexible circuit board according to some embodiments of the present disclosure;

FIG. 14 is a schematic diagram illustrating an exemplary second flexible circuit board according to some other embodiments of the present disclosure; and

FIG. 15 is a schematic diagram illustrating an internal structure of an exemplary ultrasound probe according to some other embodiments of the present disclosure.

In the figures, 100 represents a piezoelectric layer, 200 represents a backing layer, 300′ represents a first connector, 300 represents a sound head flexible circuit board, 310 represents a sound head circuit board body, 320 represents a connecting portion, 321 represents a first connecting segment, 322 represents a second connecting segment, 323 represents a third connecting segment, 324 represents a connecting contact point, 400′ represents a second connector, 400 represents a catheter flexible circuit board, 410 represents a catheter circuit board body, 420 represents a lead-out segment, 430 represents a first flexible circuit board, 431 represents a first transmission end, 440 represents a second flexible circuit board, 441 represents a second transmission end, 450 represents a first portion, 460 represents a second portion, 470 represents a catheter, 510 represents an integrated circuit, and 520 represents an electronic component.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

As shown in the present disclosure and claims, the words “one,” “a,” “a kind” and/or “the” are not especially singular but may include the plural unless the context expressly suggests otherwise. In general, the terms “comprise,” “comprises,” “comprising,” “include,” “includes,” and/or “including,” merely prompt to include operations and elements that have been clearly identified, and these operations and elements do not constitute an exclusive listing. The methods or devices may also include other operations or elements.

In the description of the present disclosure, it is to be understood that where such terms as “center,” “longitudinal,” “transverse,” “length,” “width,” “thickness,” “top”, “bottom,” “front,” “back,” “left,”, “right,” “vertical,” “horizontal,” “top,” “bottom,” “inner,” “outer,” “clockwise,” “counterclockwise,” “axial,” “radial,” “circumferential,” etc., which terms indicate an orientation or positional relationship based on that shown in the accompanying drawings relationships, and are intended only to facilitate the description of the present disclosure and to simplify the description, and not intended to indicate or imply that the device or element referred to must have a particular orientation, be constructed and operated in a particular orientation, and therefore are not to be construed as a limitation of the present disclosure.

In addition, the terms “first” and “second,” if present, are used for descriptive purposes only and are not to be understood as indicating or implying relative importance or implicitly specifying the number of technical features indicated. Thus, the feature defined with “first” and “second” may expressly or implicitly include at least one such feature. In the description of the present disclosure, where the term “plurality” occurs, “plurality” means at least two, e.g., two, three, etc., unless otherwise expressly and specifically limited.

In the present disclosure, unless otherwise expressly provided and limited, the terms “mounting,” “connecting,” “coupling,” “fixing,” etc., are to be broadly construed. For example, it may be a fixed connection, a removable connection, or a one-piece connection. It may be a mechanical connection or an electrical connection. It may be a direct connection or an indirect connection through an intermediate medium. It may be a connection within two components or between two components. It may be a connection between two components or between two components. It may be a connection between two components. It may be a connection between two components or between two components. elements in an internal connection or in an interactive relationship between the two elements, unless otherwise expressly limited. To one of ordinary skill in the art, the specific meanings of the above terms in the present disclosure may be understood on a case-by-case basis.

In the present disclosure, unless otherwise expressly provided and limited, if there is an occurrence of the first feature “over” or “under” the second feature, and other similar descriptions, the meaning may be that the first and second features are in direct contact or indirect contact through an intermediate medium. Moreover, the first feature being “above” and “over” may be that the first feature is directly above or diagonally above the second feature, or simply that the first feature is horizontally higher than the second feature. The first feature being “below,” “beneath,” and “underneath” the second feature may be that the first feature is directly below or diagonally below the second feature, or simply that the first feature has a smaller horizontal height than the second feature.

It should be noted that if a component is said to be “fixed to” or “provided on” another component, it may be directly on the other element or there may be a centered element. If an element is referred to as “connected” to another element, it may be directly connected to the other element or there may be both centered elements. If any, the terms “vertical,” “horizontal,” “up,” “down,” “left,” “right” and similar expressions used in the present disclosure are for illustrative purposes only and do not represent the only implementation method.

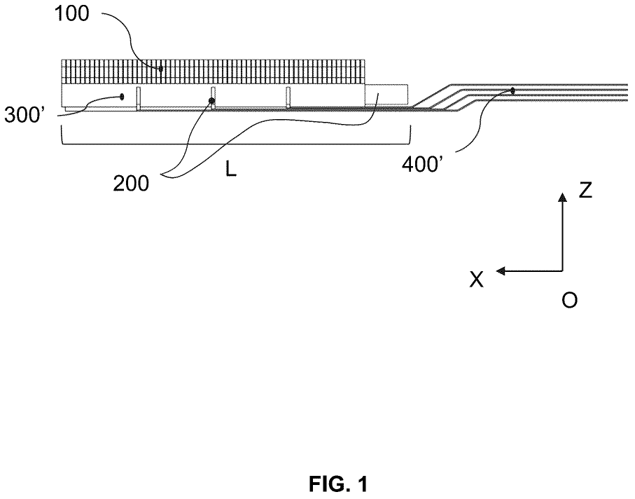

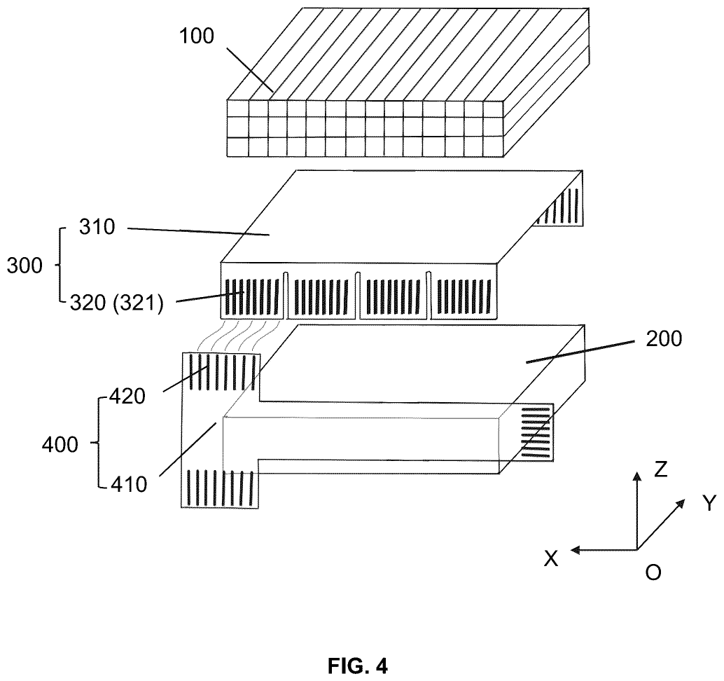

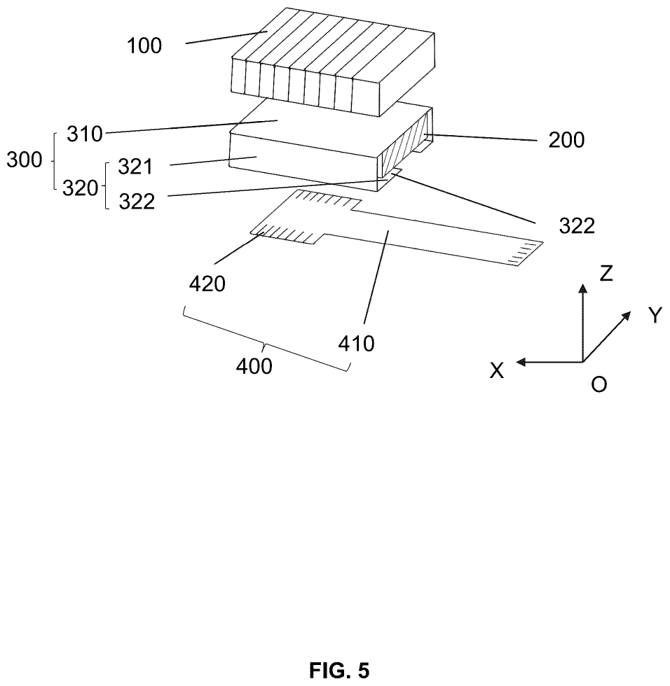

FIG. 1 is a schematic diagram illustrating an internal structure of an exemplary ultrasound probe according to some embodiments of the present disclosure. FIG. 4 is a schematic diagram illustrating an internal disassembled structure of an exemplary ultrasound probe according to some other embodiments of the present disclosure. FIG. 5 is a schematic diagram illustrating an internal disassembled structure of an exemplary ultrasound probe according to some other embodiments of the present disclosure.

As shown in FIG. 1, the ultrasound probe includes a piezoelectric layer 100, a backing layer 200, a first connector 300′, and a second connecter 400′.

The piezoelectric layer 100 is configured to convert an electrical signal into an ultrasonic signal by vibration. In some embodiments, such as FIGS. 4 and 5, the piezoelectric layer 100 includes a plurality of array elements sequentially arranged along a length direction of the backing layer 200 (e.g., the OX direction in the figures). The effective length direction of each array element (the OY direction in the figure) is the same as a width direction (OY direction) of the backing layer 200. The effective length of an array element along the effective length direction is correlated to the imaging performance of the entire ultrasound probe, therefore the effective length of the array element cannot be reduced.

In some embodiments, a transducer of the ultrasound probe may further include an acoustic lens (not shown in the figures) and an acoustic matching layer (not shown in the figures) stacked sequentially on the piezoelectric layer 100. The acoustic matching layer is closer to the piezoelectric layer 100 than the acoustic lens.

In some embodiments, the backing layer 200 may be rectangular, including a front side, a backside, and side walls connecting the front side and the backside. The backing layer 200 is in a laminated arrangement with the piezoelectric layer 100, as shown in FIG. 1. In some embodiments, a direction of the laminated arrangement may be understood to be a height direction or a thickness direction of the piezoelectric layer 100 and/or the backing layer 200. For example, the piezoelectric layer 100 and the backing layer 200 are in the laminated arrangement along the OZ direction, as shown in FIG. 1. In some embodiments, the front side of the backing layer 200 refers to a side that is close to and faces the piezoelectric layer 100. The backside of the backing layer 200 refers to a side that is away from the piezoelectric layer 100 and opposite to the front side of the backing layer 200. For example, the piezoelectric layer 100 is disposed above the front side of the backing layer 200. In some embodiments, the backside of the piezoelectric layer 100 refers to a side that is close to and faces the backing layer 200. The front side of the piezoelectric layer 100 refers to a side that is away from the backing layer 200 and opposite to the backside of the piezoelectric layer 100. The backing layer 200 is configured to counteract vibrations on the backside of the piezoelectric layer 100.

The first connector 300′ is disposed between the piezoelectric layer 100 and the backing layer 200. In some embodiments, a length of the first connector 300′ may be equal to a length of the piezoelectric layer 100. In some embodiments, the length of the first connector 300′ may be no greater than the length of the backing layer 200. In some embodiments, a width of the first connector 300′ may be no less than the width of the piezoelectric layer 100. In some embodiments, the width of the first connector 300′ may be no less than the width of the backing layer 200. In some embodiments, a projection of the first connector 300′ may overlap a projection of the piezoelectric layer 100 in an opposite direction of the OZ direction shown in FIG. 1. In some embodiments, the projection of the first connector 300′ may overlap a projection of the backing layer 200 in the OZ direction shown in FIG. 1. The first connector 300′ is electrically connected to the piezoelectric layer 100. One end of the second connector 400′ is connected to the first connector 300′ and the other end is connected to a host (e.g., a connector in the host). The first connector 300′ and the second connector 400′ are configured to realize signal transmission between the piezoelectric layer 100 and the host.

The ultrasonic signal from the piezoelectric layer 100 is transmitted to the host via the first connector 300′ and the second connector 400′ in sequence. The first connector 300′ is constructed as a flexible circuit board, which eliminates the need to provide an adapter board, an application specific integrated circuit (ASIC), and an electronic component cooperating with the ASIC, and reduces the count of the electronic component at a front end of the ultrasound probe and shortens a length of a rigid portion at the front end of the ultrasound probe (e.g., as shown by “L” in FIG. 1), improving the maneuverability of the ultrasound probe. In some embodiments, the first connector 300′ may be a flexible circuit board, and the second connector 400′ may be a wired cable, etc.

In some embodiments, the first connector 300′ and the second connector 400′ may both be constructed as flexible printed circuit boards (FPC). The flexible circuit boards not only provide excellent electrical properties to transmit the ultrasonic signal from the piezoelectric layer 100 to the host, but also allow for smaller ultrasound probe sizes.

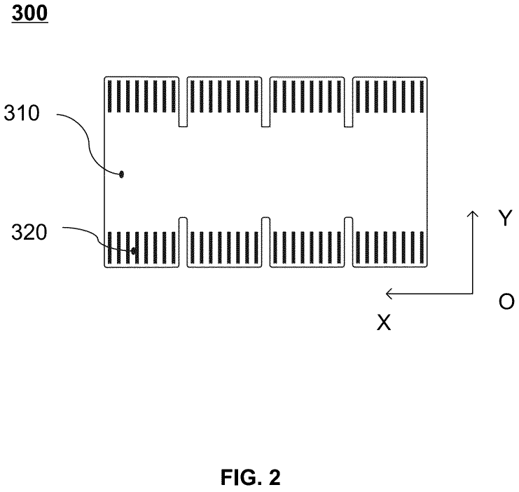

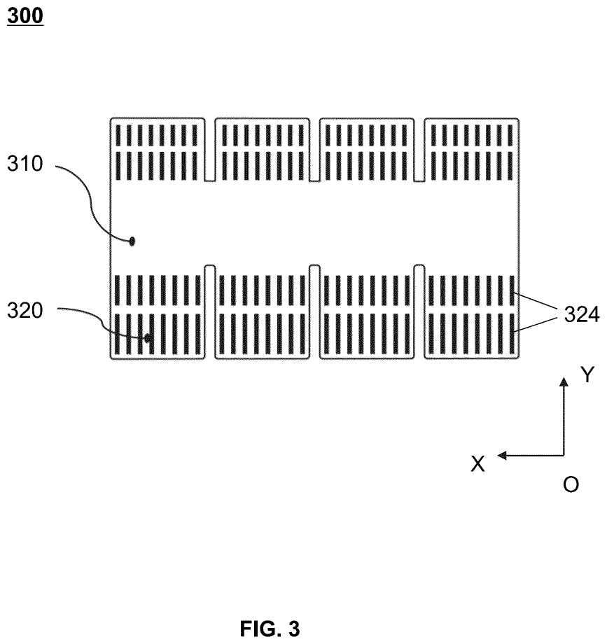

FIG. 2 is a schematic diagram illustrating an exemplary sound head flexible circuit board according to some embodiments of the present disclosure. FIG. 3 is a schematic diagram illustrating an exemplary sound head flexible circuit board according to some other embodiments of the present disclosure. In some embodiments, the first connector 300′ is or includes a sound head flexible circuit board 300. As shown in FIG. 2 and FIG. 3, the sound head flexible circuit board 300 may include a sound head circuit board body 310 and a connecting portion 320 disposed on a periphery of the sound head circuit board body 310.

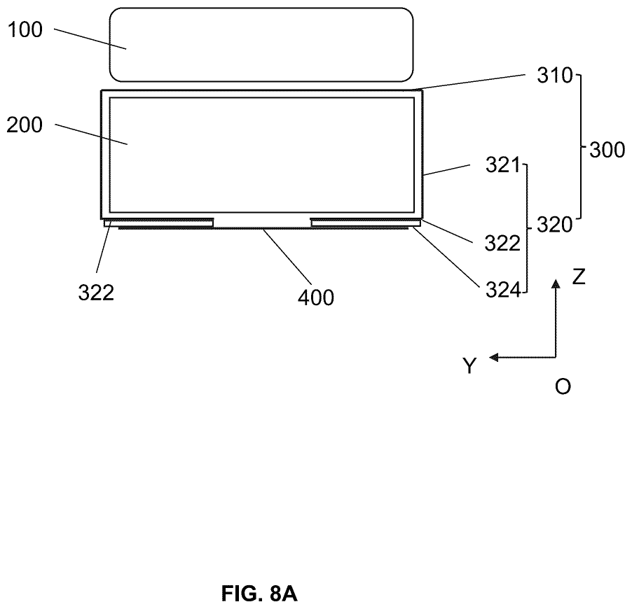

The sound head circuit board body 310 and the connecting portion 320 may be integrally molded, or the connecting portion 320 is connected to the sound head circuit board body 310 after being separately molded. The connecting methods include soldering, bonding, binding, etc. In some embodiments, the sound head circuit board body 310 and the connecting portion 320 may be in the same plane as shown in FIG. 2 and FIG. 3. In some embodiments, the sound head circuit board body 310 and the connecting portion 320 may be in different planes. For example, as shown in FIG. 4, the connecting portion 320 is arranged along the periphery of the sound head circuit board body 310 and is bent relative to the sound head circuit board body 310. In some embodiments, as shown in FIG. 4, the sound head circuit board body 310 may be disposed between the piezoelectric layer 100 and the backing layer 200. In some embodiments, the sound head circuit board body 310 may be in contact with the piezoelectric layer 100 and the backing layer 200. An upper surface of the sound head circuit board body 310 may be in contact with a lower surface of the piezoelectric layer 100. A lower surface of the sound header circuit board body 310 may be in contact with an upper surface of the backing layer 200. In some embodiments, a length of the sound head circuit board body 310 may be equal to a length of the piezoelectric layer 100. In some embodiments, the length of the sound header circuit board body 310 may be no greater than a length of the backing layer 200. In some embodiments, a width of the sound header circuit board body 310 may be equal to the width of the piezoelectric layer 100 and the backing layer 200. In some embodiments, the connecting portion 320 may be connected to the second connector 400′. The sound head flexible circuit board 300 may be connected to the second connector 400′ via the connecting portion 320. Descriptions relating to the connection between the connecting portion 320 and the second connector 400′ may be found elsewhere in the present disclosure (e.g., FIGS. 8A-8C, FIGS. 11-12, and their related descriptions) and are not be repeated here.

In some embodiments, as shown in FIGS. 1, 2, and 3, length directions of the piezoelectric layer 100, the backing layer 200, the first connector 300′, the body of the acoustic header circuit board 310, and the second connector 400′ (e.g., a catheter flexible circuit board 400) may be the OX direction or an opposite direction of the OX direction. Width directions of the piezoelectric layer 100, the backing layer 200, the first connector 300′, the sound head circuit board body 310, and the second connector 400′ (e.g., the catheter flexible circuit board 400) may be the OY direction or an opposite direction of the OY direction. A plurality of connecting portions 320 are disposed on at least one end of the sound head circuit board body 310 along the width direction. For example, as shown in FIG. 2 and FIG. 3, the sound head circuit board body 310 is provided with a plurality of connecting portions 320 at each end along the width direction. As another example, when the width of the piezoelectric layer 100 is small, the connecting portion 320 may be provided at one end of the sound head circuit board body 310 along the width direction.

In some embodiments, the plurality of connecting portions 320 are sequentially arranged along the length direction of the piezoelectric layer 100. In some embodiments, the plurality of connecting portions 320 are sequentially arranged in at least one row and each row of the at least one row is along a length direction of the piezoelectric layer 100. In some embodiments, the plurality of connecting portions 320 may be arranged in a plurality of rows along the length direction of the piezoelectric layer 100 at the same end of the sound header circuit board body 310 along the width direction. In some embodiments, two adjacent connecting portions 320 among the plurality of connecting portions 320 at a same end of the sound head circuit board body 310 along the width direction of the sound head circuit board body 310 are disconnected. In some embodiments, a plurality of connecting portions 320 at a same end of the sound head circuit board body 310 along the width direction may be connected, or disconnected by a pad. In some embodiments, the plurality of connecting portions 320 may be provided in pairs. Two connecting portions 320 in a same pair of connecting portions are disposed at two ends of the sound head circuit board body 310 along the width direction. Compared with arranging the connecting portions 320 at only one end of the sound head circuit board body 310 along the width direction, arranging the connecting portions 320 in pairs at both ends of the sound head circuit board body 310 along the width direction may shorten the total length of the sound head flexible circuit board 300, which may further shorten the length of the ultrasound probe. The two connecting portions 320 provided in pairs may be arranged opposite to each other along the width direction, or may be arranged in a staggered manner, which is not limited herein.

In some embodiments, as shown in FIG. 4, the connecting portions 320 may include a first connecting segment 321. In some embodiments, the first connecting segment 321 and the sound head circuit board body 310 may be integrally molded. In some embodiments, the first connecting segment 321 may be connected to the sound head circuit board body 310. Connection methods may include soldering, bonding, binding, etc. The first connecting segment 321 may be provided on the periphery of the sound head circuit board body 310 and bend away from the piezoelectric layer 100 (e.g., the opposite direction of the OZ direction in FIG. 4). In some embodiments, the first connecting segment 321 may extend in a direction away from the piezoelectric layer 100 (e.g., the opposite direction of the OZ direction in FIG. 4) along a side wall of the backing layer 200. In this way, the size (e.g., the width) of the ultrasound probe is reduced. In some embodiments, the first connecting segment 321 may be in contact with a side wall of the backing layer 200. The sound head flexible circuit board 300 and the second connector 400′ may be connected by the first connecting segment 321.

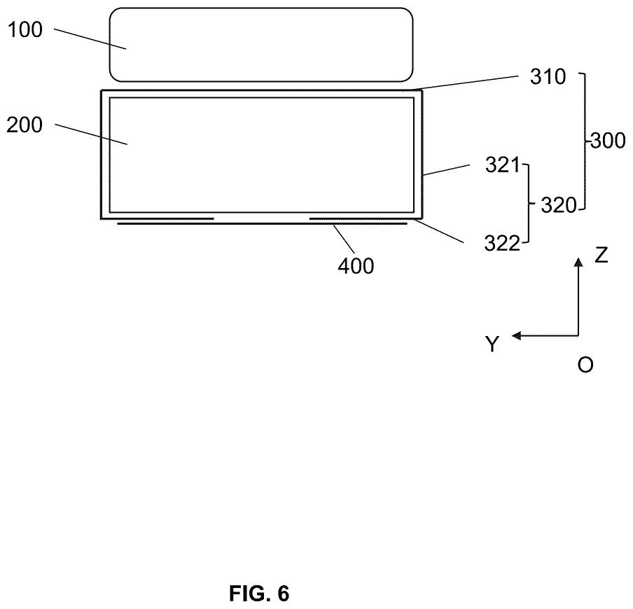

FIG. 5 is a schematic diagram illustrating an internal disassembled structure of an exemplary ultrasound probe according to some other embodiments of the present disclosure. FIG. 6 is a left view of the ultrasound probe shown in FIG. 5 after assembly.

In some embodiments, as shown in FIG. 5 and FIG. 6, the connecting portions 320 may further include a second connecting segment 322. The second connecting segment 322 is connected to the first connecting segment 321 and extends over a backside of the backing layer 200 away from the piezoelectric layer 100. In some embodiments, the second connecting segment 322 may be in contact with a lower bottom surface of the backing layer 200. In some embodiments, the second connecting segment 322 may be connected to the first connecting segment 321 in a manner such as, soldering, bonding, or binding connections. In some embodiments, the second connecting segment 322 and the first connecting segment 321 may be integrally molded. As shown in FIG. 5 and FIG. 6, the sound head circuit board body 310, the first connecting segment 321, and the second connecting segment 322 may be wrapped around the backing layer 200, which may improve an internal stability of the structure of a sound head portion of the ultrasound probe to avoid wobbling. In some embodiments, the second connector 400′ may be connected to the first connecting segment 321 and/or the second connecting segment 322. For example, the sound head flexible circuit board 300 and the second connector 400′ may be connected by the first connecting segment 321. As another example, as shown in FIGS. 5 and 6, the sound head flexible circuit board 300 and the second connector 400′ (e.g., the catheter flexible circuit board 400) may be connected on a backside of the backing layer 200 via the second connecting segment 322, which facilitates a reduction in the size of the entire sound head portion and improves the maneuverability of the sound head portion. As another example, the sound head flexible circuit board 300 and the second connector 400′ may be connected by the first connecting segment 321 and the second connecting segment 322 to improve the stability of the connection.

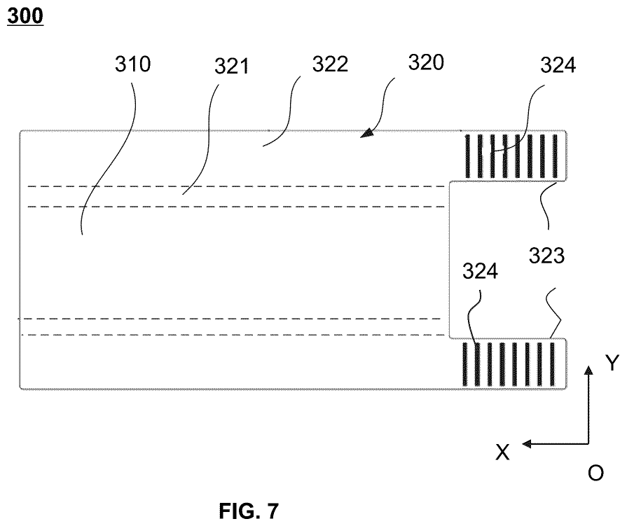

FIG. 7 is a schematic diagram illustrating an exemplary sound head flexible circuit board 300 according to some other embodiments of the present disclosure. As shown in FIG. 7, the connecting portions 320 may include a third connecting segment 323 provided at one end of the second connecting segment 322 along a length direction of the second connecting segment 322 (e.g., the OX direction in FIG. 7). In some embodiments, the second connecting segment 322 is connected to the third connecting segment 323 via soldering, bonding, binding, etc. In some embodiments, the third connecting segment 323 and the second connecting segment 322 may be integrally molded. The sound head flexible circuit board 300 and the second connector 400′ (e.g., the catheter flexible circuit board 400) may be connected by the third connection segment 323.

In some embodiments, one or more connecting contact points 324 (which may be referred to as first connecting contacts) may be provided on the connecting portions 320. The connecting portions 320 may be connected to the second connector 400′ via the connecting contact points 324. In some embodiments, at least one of the first connecting segment 321, the second connecting segment 322, and the third connecting segment 323 may be provided with the connecting contact point(s) 324. For example, the sound head flexible circuit board 300 and the second connector 400′ may be connected via connecting contact points 324 on the first connecting segment 321. As another example, the sound head flexible circuit board 300 and the second connector 400′ may be connected via connecting contact points 324 on the second connecting segment 322. As yet another example, the sound head flexible circuit board 300 and the second connector 400′ may be connected via connecting contact points 324 on the third connecting segment 323. As yet another example, the sound head flexible circuit board 300 and the second connector 400′ may be connected via the connecting contact points 324 on the first connecting segment 321 and the connecting contact points 324 on the second connecting segment 322.

FIG. 8A is a sectional view in a width direction of an exemplary ultrasound probe according to some other embodiments of the present disclosure. FIG. 8B is a sectional view in a width direction of an exemplary ultrasound probe according to some other embodiments of the present disclosure. FIG. 8C is a sectional view in a width direction of an exemplary ultrasound probe according to some other embodiments of the present disclosure. As shown in FIG. 8A, in some embodiments, the connecting contact points 324 may be disposed on a side of the second connecting segment 322 away from the backing layer 200. One end of the second connector 400′ (e.g., catheter flexible circuit board 400) is disposed below the backing layer 200 and the sound head flexible circuit board 300 and is electrically connected to the second connecting segment 322 via the connecting contact points 324.

As shown in FIG. 8B, in some embodiments, the connecting contact points 324 may be provided on a side of the second connecting segment 322 proximate to the backing layer 200. One end of the second connector 400′ (e.g., catheter flexible circuit board 400) is disposed between the backing layer 200 and the sound head flexible circuit board 300, electrically connected to the second connecting segment 322 via the connecting contact points 324.

As shown in FIG. 8C, in some embodiments, second connecting segments 322 are provided on two ends of the sound head circuit board body 310 along the width direction of the sound head circuit board body 310, a side of the second connecting segment 322 away from the backing layer 200 on the one end may be provided with connecting contact points 324, and a side of the second connecting segment 322 close to the backing layer 200 on the other end may be provided with connecting contact points 324. Among the two sides of one end of the second connector 400′ (e.g., the catheter flexible circuit board 400) along the width direction, one side is disposed below the backing layer 200 and the sound head flexible circuit board 300, the other side is disposed between the backing layer 200 and the sound head flexible circuit board 300, and the two sides are electrically connected to the second connecting segment 322 via the connecting contact points 324.

In some embodiments, the connecting portion 320 is provided with one or more rows of connecting points 324 along the width direction. For example, as shown in FIG. 3, two rows of connecting contact points 324 are provided on each of the connecting portions 320 provided on two ends of the sound head circuit board body 310 along the width direction (OY direction) of the sound head circuit board body 310. Each row of connecting contact points 324 includes a plurality of connecting contact points 324 disposed along a length direction (OX direction). In some embodiments, a count of rows and a count of connecting contact points 324 provided on each connecting portion 320 on the two ends of the sound head circuit board body 310 along the width direction may be the same or different. For example, in the connecting portions 320 provided on the two ends of the sound head circuit board body 310 along the width direction, the connecting portion 320 on the one end may be provided with connecting contact points 324, and the connecting portion 320 on the other end may be not provided with connecting contact points 324. Providing at least two rows of connecting contact points 324 may reduce the connection difficulty between the sound head flexible circuit board 300 and the second connector 400′.

In some embodiments, the sound head flexible circuit board 300 may be connected to the second connector 400′ by other means, for example, using a pad. In some embodiments, at least one of the connecting contact points 324 may be replaced with a pad. For example, the connecting portion 320 may be provided with one or more pads. The connecting portion 320 may be connected to the second connector 400′ via the pads. The pads may be provided on a side of the second connecting segment 322 near the backing layer 200 and/or provided on a side of the second connecting segment 322 away from the backing layer 200. For example, the connecting portion 320 is provided with one or more rows of pads along the width direction. In some embodiments, when the sound head flexible circuit board 300 is connected to the second connector 400′ via the first connecting segment 321, the first connecting segment 321 may be provided with pads and/or connection contact points 324. In some embodiments, when the sound head flexible circuit board 300 and the second connector 400′ are connected via the second connecting segment 322, the second connecting segment 322 may be provided with pads and/or connecting contact points 324. In some embodiments, the sound head flexible circuit board 300 and the second connector 400′ are connected via the third connecting segment 323, the third connecting segment 323 may be provided with pads and/or connecting contact points 324. In some embodiments, the sound head flexible circuit board 300, the first connecting segment 321, and second connecting segment 322 of the second connector 400′ may have the same or different settings when connected via the first connecting segment 321 and the second connecting segment 322. For example, the first connecting segment 321 and the second connecting segment 322 may both be provided with pads and/or connecting contact points 324. As another example, the first connecting segment 321 may be provided with the pads while the second connection segment 322 may be provided with the connecting contact points 324. As yet another example, the first connecting segment 321 may be provided with the connecting contact points 324 while the second connecting segment 322 may be provided with the pads.

Signals of each channel of the piezoelectric layer 100 need to be outputted to the host separately. Therefore, a total count of channels led out from the connecting portion 320 on the sound head flexible board 300 corresponds to (i.e., is equal to) the count of channels of the piezoelectric layer 100 so as to facilitate signal transmission for each channel. In some embodiments, a single connecting contact point 324 transmits signals of one channel. For example, the ultrasound probe (or piezoelectric layer 100) includes 128 channels, and accordingly, 128 connecting contact points 324 (which may be referred to as the first connecting contacts) are provided on the connecting portion 320 of the sound head flexible circuit board 300. A count of cables of the second connector 400′ is 128, and the 128 cables are connected one-to-one with the 128 connecting contact points 324. In some embodiments, the second connector 400′ is a catheter flexible circuit board 400, and 128 connecting contact points (which may be referred to as second connecting contact points) may be provided on a lead-out segment 420 of the catheter flexible circuit board 400. The 128 second connecting contact points are connected one-to-one with 128 connecting contact points 324 (which may be referred to as first connecting contact points) on the connecting portion 320.

In some embodiments, the ultrasound probe may include a first carrier unit (not shown in the figure). In some embodiments, the first carrier unit may be a signal processing unit. In some embodiments, the first carrier unit may be disposed on the sound head flexible circuit board 300. In some embodiments, the first carrier unit may be provided on another component independent from the acoustic head flexible circuit board 300. The first carrier unit may be electrically connected to the piezoelectric layer 100 and the sound head flexible circuit board 300. In some embodiments, the first carrier unit may perform frequency division processing on a plurality of electrical signals outputted by the piezoelectric layer 100 to obtain a plurality of divided electrical signals, and transmit the plurality of divided electrical signals to the sound head flexible circuit board 300. For example, the connecting contact points 324 on the connecting portion 320 transmit the plurality of divided electrical signals to the host via the second connector 400′. In some embodiments, the plurality of divided electrical signals have different carrier frequency bands. Dividing the frequency of the electrical signals by the first carrier unit allows the same connecting contact point 324 to transmit a plurality of electrical signals of different frequency bands at the same time, which in turn allows the count of channels to be reduced, and reduces the volume of the catheter, or avoids the limitation caused by the physical size of the cable. In some embodiments, the frequency bands of the electrical signals transmitted by the different connecting contact points 324 may be the same. For example, the ultrasound probe includes 128 channels, and the plurality of electrical signals are frequency-division-multiplexed by the first carrier unit such that a single connecting contact point 324 may transmit electrical signals of 2 channels. 64 connecting contact points 324 (which may be referred to as first connecting contact points) on the connecting portion 320 of the sound head flexible circuit board 300 may fulfill the requirement. Correspondingly, the count of cables of the second connector 400′ may be 64, and the 64 cables are connected one-to-one with the 64 connecting contact points 324. In some embodiments, the second connector 400′ is a catheter flexible circuit board 400, and 64 connecting contact points (which may be referred to as second connecting contact points) may be provided on the lead-out segment 420 of the catheter flexible circuit board 400. The 64 second connecting contact points are connected one-to-one with the 64 connecting contact points 324 (which may be referred to as first connecting contact points) on the connecting portion 320.

In some embodiments, the ultrasound probe may include a second carrier unit (not shown in the figure). In some embodiments, the second carrier unit may be a signal processing unit. In some embodiments, the second carrier unit may be disposed on the sound head flexible circuit board 300. In some embodiments, the second carrier unit may be provided on another component independent from the sound head flexible circuit board 300. The second carrier unit may be electrically connected to the piezoelectric layer 100 and the sound head flexible circuit board 300. In some embodiments, the second carrier unit may transmit a plurality of electrical signals outputted from the piezoelectric layer 100 to the sound head flexible circuit board 300 sequentially during different transmission time periods. The connecting contact points 324 on the connecting portion 320 may transmit the plurality of electrical signals to the host via the second connector 400′. For each transmission time period, the connecting contact point 324 transmits only one electrical signal. Time-division multiplexing of the electrical signals by the second carrier unit allows the same connecting contact point 324 to transmit the plurality of electrical signals, which in turn allows for a reduction in the count of channels, a reduction in the volume of the catheter, or avoids the limitation caused by the physical size of the cables. For example, the ultrasound probe includes 128 channels, and time-division multiplexing of the plurality of electrical signals by the second carrier unit allows a single connecting contact point 324 to transmit electrical signals of 2 channels at two different transmission times. 64 connecting contact points 324 (which may be referred to as first connecting contact points) on the connecting portion 320 of the sound head flexible circuit board 300 may fulfill the requirement. Correspondingly, the count of cables of the second connector 400′ may be 64, and the 64 cables are connected one-to-one with the 64 connecting contact points 324. In some embodiments, the second connector 400′ is a catheter flexible circuit board 400, and 64 connecting contact points (which may be referred to as second connecting contact points) may be provided on the lead-out segment 420 of the catheter flexible circuit board 400. The 64 second connecting contact points are connected one-to-one with the 64 connecting contact points 324 (which may be referred to as first connecting contact points) on the connecting portion 320.

The second connector 400′ can be constructed as a flexible circuit board, the existing coaxial cable. In some embodiments, the second connector 400′ may be configured as a flexible circuit board, replacing the existing coaxial cables. This can avoid the limitations on the number of coaxial cables due to the restricted inner space within the catheter, which could otherwise limit the number of multi-channel transducers. In some embodiments, the second connector 400′ may include at least one catheter flexible circuit board 400. The at least one catheter flexible circuit board 400 includes at least one flexible circuit board disposed in at least one catheter. The at least one catheter flexible circuit board 400 may be electrically connected to the first connector 300′.

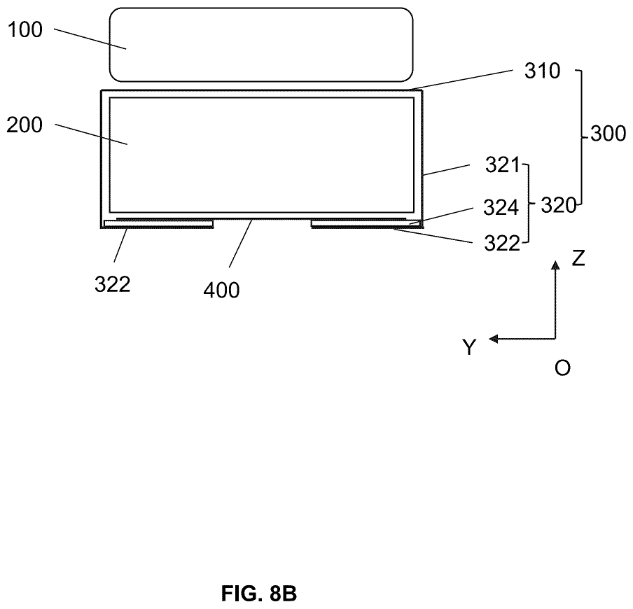

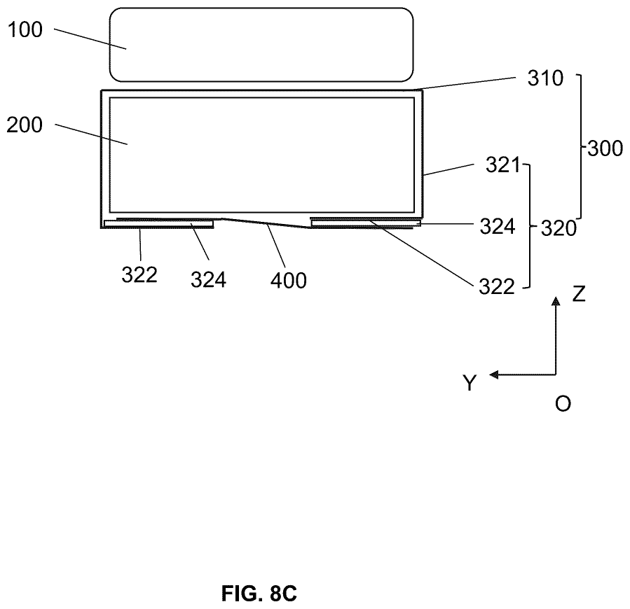

FIG. 9 is a schematic diagram illustrating an exemplary catheter flexible circuit board according to some embodiments of the present disclosure. FIG. 10 is a schematic diagram illustrating an exemplary catheter flexible circuit board according to some other embodiments of the present disclosure.

In some embodiments, as shown in FIG. 9 and FIG. 10, for each of the at least one catheter flexible circuit board 400, the catheter flexible circuit board 400 may include a catheter circuit board body 410 and a lead-out segment 420. The lead-out segment 420 may be located at at least one end of the catheter circuit board body 410 along a width direction of the catheter circuit board body 410 (such as the OY direction in the figure). As shown in FIG. 9, the catheter circuit board body 410 is provided with lead-out segments 420 at two ends of the catheter circuit board body 410 along the width direction of the catheter circuit board body 410. As shown in FIG. 10, the catheter circuit board body 410 is provided with one lead-out segment 420 at one end along the width direction of the catheter circuit board body 410.

The first connector 300′ (e.g., the sound head flexible circuit board 300) and the second connector 400′ (e.g., the catheter flexible circuit board 400) may be electrically connected to the lead-out segment 420 via the connecting portion 320. In some embodiments, there are multiple lead-out segments 420 and multiple connecting portions 320 that are provided in correspondence. In other words, each lead-out segment 420 has a corresponding connecting portion 320, and the lead-out segment 420 and the corresponding connecting portion 320 are electrically connected to each other. The count of electrical signals led out from the lead-out segment 420 is equal to the count of electrical signals drawn out of the connecting portion 320, and the two are connected one-to-one in the order of leading-out or the order of arrangement. That is, the signals of each channel may be transmitted to the connector through the sound head flexible circuit board 300, and the catheter flexible circuit board 400 in sequence.

In some embodiments, the count of the catheter flexible circuit board 400 is at least one or at least two. As shown in FIG. 9, each end of the catheter circuit board body 410 along the width direction of the piezoelectric layer 100 (e.g., in the OY direction in the FIG.) is provided with one lead-out segment 420, and the two lead-out segments 420 are connected to a pair of connecting portions 320 in one-to-one correspondence (i.e., each lead-out segment 420 is connected to one connecting portion 320).

In some embodiments, the two lead-out segments 420 are located at the two sides of an end portion of the catheter circuit board body 410, i.e., the catheter flexible circuit board 400 is a T-shaped structure. In actual use, two lead-out segments 420 on the catheter circuit board body 410 are connected one-to-one with a pair of connecting portions 320 (e.g., a pair of second connecting segment 322 or a pair of third connecting segment 323) when the connecting portions 320 (e.g., a pair of second connecting portions 322 or a pair of third connecting segment 323) are folded over to the backside of the backing layer 200. A plurality of catheter flexible circuit boards 400 need to be provided, and the plurality of catheter flexible circuit boards 400 are sequentially stacked in a staggered manner along the length direction of the piezoelectric layer 100 (e.g., the OY direction in the figures). In such cases, the lead-out segments 420 of different catheter flexible circuit boards 400 are arranged staggerly.

Specifically, in the case where four pairs of connecting portions 320 (e.g., four pairs of first connection segments 311) are provided on the sound head flexible circuit board 300, for example, two lead-out segments 420 are provided at an end of each catheter circuit board body 410 along the width direction (e.g., in the OY direction in the FIG.) of the piezoelectric layer 100, i.e., there is a need for four catheter flexible circuit boards 400. The four catheter flexible circuit boards 400 are sequentially staggered and stacked along the length direction (e.g., the OX direction in the figure), such that two lead-out segments 420 on each catheter flexible circuit board 400 are connected to a pair of connecting portions 320 (e.g., the first connecting segments 311) in a one-to-one correspondence.

In some embodiments, the count of catheter flexible circuit boards 400 is more than 1. As shown in FIG. 10, the catheter circuit board body 410 is provided with a lead-out segment 420 at one end along the width direction of the piezoelectric layer 100 (e.g., in the OY direction in the figure). In such cases, a pair of connecting portions 320 are connected to the lead-out segments 420 of two catheter flexible circuit boards 400.

In some embodiments, only one lead-out segment 420 is provided on the catheter flexible circuit board 400, and the catheter flexible circuit board 400 is an L-shaped structure. In actual use, the count of the catheter flexible circuit board 400 is the same as the count of the connecting portion 320, the plurality of catheter flexible circuit boards 400 are disposed in two rows along the width direction (e.g., the OY direction in the FIG.) of the piezoelectric layer 100, and the plurality of catheter flexible circuit boards 400 in each row is staggerly stacked in sequence along the length direction (e.g., the OX direction in the FIG.) of the piezoelectric layer 100.

In some embodiments, the catheter circuit board body 410 is provided with a plurality of lead-out segments 420, e.g., at least two lead-out segments 420, at each end of the catheter circuit board body 410 along the width direction of the piezoelectric layer 100 (e.g., the OY direction in the figure). The plurality of lead-out segments 420 may be sequentially connected. Alternatively, two adjacent lead-out segments 420 located at the at the same end along the width direction of the catheter circuit board body 410 are disconnected. In some embodiments, the plurality of lead-out segments 420 being sequentially connected refers to that the substrates of the plurality of lead-out segments 420 are connected. In some embodiments, two adjacent lead-out segments 420 are disconnected refers to that the substrates of the two adjacent lead-out segments 420 are spaced apart without affecting the electrical connection between the adjacent lead-out segments 420 and the first connector 300′ (or connecting portion 320). Specifically, when the plurality of lead-out segments 420 are connected sequentially, pads disposed on the two adjacent lead-out segments 420 are spaced apart in order to facilitate that the plurality of lead-out segments 420 are connected to the plurality of connecting portions 320 in a one-to-one correspondence. When the two adjacent connecting portions 320 at the same end along the width direction of the sound head circuit board body 310 are disconnected from each other and a plurality of lead-out segments 420 are connected sequentially, in order to facilitate one-to-one connection of the plurality of lead-out segments 420 to the plurality of connecting portions 320, a pad may be provided on each of the lead-out segments 420 individually and adjacent pads may be disconnected from each other. In some embodiments, the catheter circuit board body 410 is provided with four interconnected lead-out segments 420 on each side along the width direction of the piezoelectric layer 100 (e.g., in the OY direction in FIG. 4), and pads on two adjacent lead-out segments 420 are spaced apart. At this time, a count of electrical signals led out from the lead-out segments 420 on the catheter flexible board 400 is equal to a count of electrical signals led out from the connecting portion 320 on the sound head flexible board 300 (such as the connecting portion 320 shown in FIG. 2 or the first connecting segment 321 shown in FIG. 4). It is also possible that each side of the catheter circuit board body 410 along the width direction (such as the OY direction in the figure) the piezoelectric layer 100 is provided with two lead-out segments 420, respectively. At this time, two catheter flexible circuit boards 400 need to be provided to enable the count of electrical signals led out from the lead-out segment 420 to be equal to the count of electrical signals led out from the connecting portion 320 on the sound head flexible circuit board 300 (e.g., the connecting portion 320 as shown in FIG. 2 or the first connecting segment 321 as shown in FIG. 4). Alternatively, the catheter flexible circuit board 400 has a structure which is a combination of the L-shaped structure and the T-shaped structure. The count and structure of the catheter flexible circuit board 400 are related to the count of channels of the piezoelectric layer 100, the count of connecting portions 320 of the sound head flexible circuit board 300, and the size of the space of the inner cavity channels of the catheter (e.g., the size of the space of the catheter), as long as the total count of the connecting portions 320 (which may be understood as at least one of the first connecting segment 321, the second connecting segment 322, or the third connecting segment 323 electrically connected with the lead-out segment 420) is equal to the total count of the lead-out segments 420, and the connecting portions 320 are one-to-one correspondingly connected to the lead-out segments 420.

In some embodiments, a connecting portion 320 may be welded, bonded, bound, or integrally molded to a corresponding lead-out segment 420.

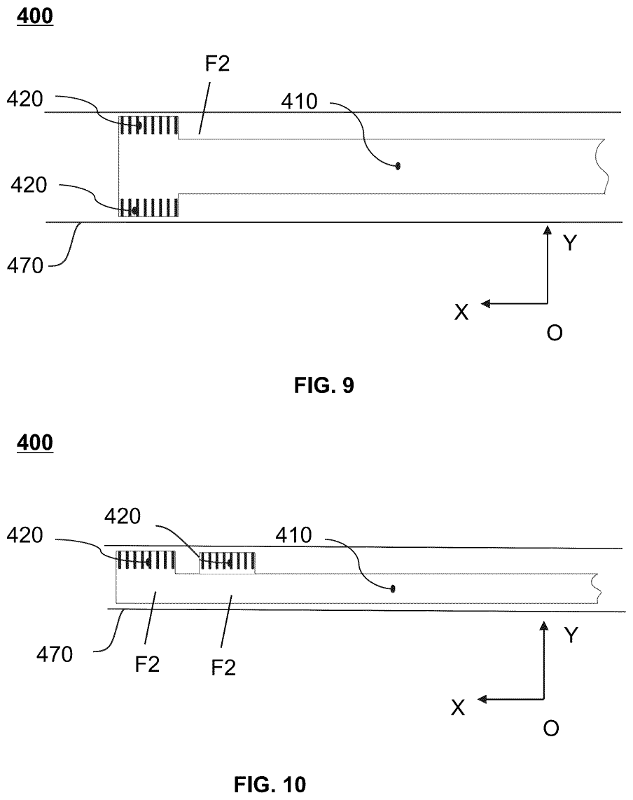

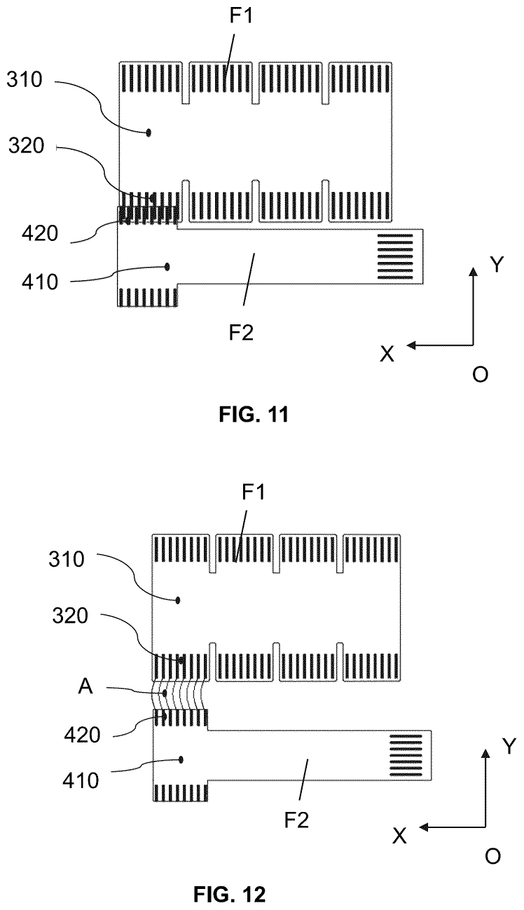

FIG. 11 is a schematic diagram illustrating a structure in which an exemplary sound head flexible circuit board is connected to an exemplary catheter flexible circuit board according to some embodiments of the present disclosure. FIG. 12 is a schematic diagram illustrating a structure in which an exemplary sound head flexible circuit board is connected to an exemplary catheter flexible circuit board catheter flexible circuit board according to another embodiment of the present disclosure. In some embodiments, pads may be provided on the connecting portion 320 and the lead-out segment 420, respectively. In some embodiments, pads may only be provided on a connecting portion 320 (e.g., the second connecting segment 322) on a backside of the backing layer 200; no pads are provided on a connecting portion 320 (e.g., the first connecting segment 321) on a sidewall of the backing layer 200. The connecting portion 320 is soldered to the lead-out segment 420 as shown in FIG. 11. As shown in FIG. 12, the connecting portion 320 is bonded or tied to the lead-out segment 420. For example, the connecting portion 320 is tied to the lead-out segment 420 by wire A. In some embodiments, the connecting portion 320 and the lead-out segment 420 may be integrally molded.

In some embodiments, two adjacent connecting portions 320 along the width direction are connected to each other, but the pads disposed on the connecting portions 320 are spaced apart from each other, thereby facilitating one-to-one connection of the pads on the connecting portions 320 to the pads on the lead-out segments 420. In some embodiments, two adjacent connecting portions 320 are connected to each other refers to that the substrates of the two adjacent connecting portions 320 are spatially connected. When two adjacent connecting portions 320 are connected to each other, the pads may be provided on each connecting portion 320 and each lead-out segment 420, respectively. In such cases, adjacent pads on adjacent connecting portions 320 are disconnected, adjacent pads on adjacent lead-out segments 420 are disconnected, which facilitates the one-to-one corresponding connection between a plurality of connecting portions 320 and a plurality of lead-out segments 420.

In some embodiments, a lead-out segment 420 may be provided with one or more connecting contact points (not shown in the figures and may be referred to as second connecting contact points). The lead-out segment 420 is connected to the first connector 300′ (e.g., the sound head flexible circuit board 300) via the second connecting contact point(s). The second connecting contact point(s) may be provided on a side of the lead-out segment 420 near the connecting portion 320. In some embodiments, at least two rows of second connecting contact points are arranged side by side along the width direction (OY direction in the figure) of the catheter circuit board body 410, and each row of the at least two rows of second connecting contact points includes a plurality of second connecting contact points arranged along the length direction (OX direction in the figure) of the catheter circuit board body 410. Providing at least two rows of the second connecting contact points may reduce the connection difficulty between the lead-out segment 420 and the connecting portion 320.

When one row of the at least two rows of second connecting contact points fails, the remaining rows of second connecting contact points may be used, increasing the utilization rate of the catheter flexible circuit board 400.

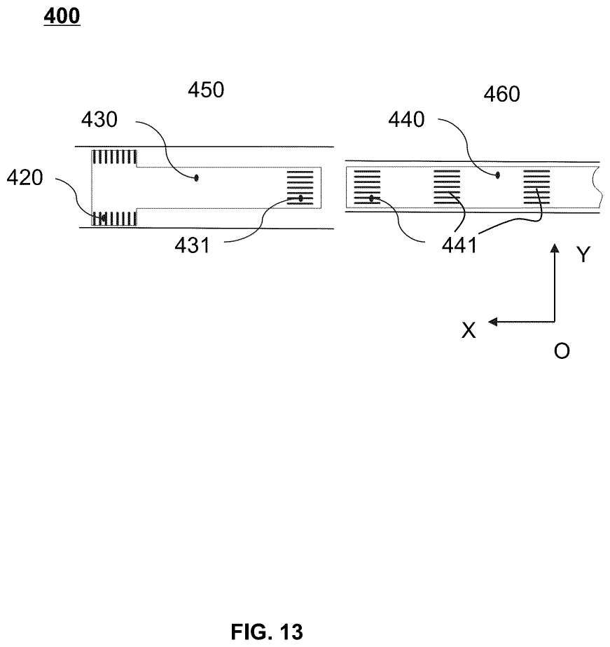

FIG. 13 is a schematic diagram illustrating structures of an exemplary first flexible circuit board and an exemplary second flexible circuit board according to some embodiments of the present disclosure.

In some embodiments, as shown in FIG. 13, the catheter flexible circuit board 400 may include a first flexible circuit board 430 and a second flexible circuit board 440 that are connected to each other. An end of the first flexible circuit board 430 away from the second flexible circuit board 440 is connected to the first connector 300′, and an end of the second flexible circuit board 440 away from the first flexible circuit board 430 is connected to the host. For example, the end of the first flexible circuit board 430 away from the second flexible circuit board 440 is connected to the connecting portion 320, and the end of the second flexible circuit board 440 away from the first flexible circuit board 430 is configured to be connected to a coupler of the host.

In some embodiments, the ultrasound probe may include a first portion (also referred to as a sound head portion) and a second portion (also referred to as a catheter portion). In some embodiments, the first portion and the second portion may be integrated. In some embodiments, the first portion and the second portion may be connected. The second portion is connected to the coupler. When in use, the first portion is placed to a designated location for scanning, and the second portion connects the first portion with the host. The catheter flexible circuit board 400 includes a first flexible circuit board 430 and a second flexible circuit board 440, the first connector 300′ and the first flexible circuit board 430 may be provided within the first portion of the ultrasound probe, and the second flexible circuit board 440 is provided within the second portion of the ultrasound probe. In some embodiments of the present disclosure, the first portion (i.e., the sound head portion) includes the piezoelectric layer 100, the backing layer 200 and the first connector 300′. When the first connector 300′ and the first flexible circuit board 430 are within the first portion of the ultrasound probe, the first portion includes the piezoelectric layer 100, the backing layer 200, the first connector 300′, and the first flexible circuit board 430. In some embodiments, the second portion (i.e., the catheter portion) includes the second connector 400′ in at least one catheter. When the first flexible circuit board 430 is within the first portion of the ultrasound probe and the second flexible circuit board 440 is within the second portion of the ultrasound probe, the second portion includes the second flexible circuit board 440 in at least one catheter, but not the first flexible circuit board 430. In some embodiments, such as in an intracardiac echocardiography catheter (ICE), the ultrasound probe is single-use. When the ultrasound probe is replaced, the first flexible circuit board 430 is replaced at the same time with the ultrasound probe, and the second flexible circuit board 440 may be connected to a first flexible circuit board in the new ultrasound probe 430 for reuse.

In some embodiments, as shown in FIG. 13, the first flexible circuit board 430 may have a first transmission end 431 and the second flexible circuit board 440 may have a plurality of second transmission ends 441. The plurality of second transmission ends 441 may be spaced sequentially along an extension direction of the second flexible circuit board 440, with the first transmission end 431 optionally connected to any one of the plurality of second transmission ends 441.

In use, the second flexible circuit board 440 has a plurality of second transmission ends 441, and each ultrasound probe is connected to only one of the plurality of second transmission ends 441 via the first transmission end 431. When the ultrasound probe is replaced, the second transmission end 441 connected to the first flexible circuit board 430 of the previous ultrasound probe may be invalidated, and at this time, it is only necessary to connect the replaced ultrasound probe to another second transmission end 441. Specifically, by providing multiple second transmission ends 441 on the second flexible circuit board 440, when the second transmission end 441 proximate to the first flexible circuit board 430 fails, the failed portion can be physically removed (cut), the next second transmission end 441 along the extension direction of the second flexible circuit board 440 can be selected as a new connecting point, thus the second flexible circuit board 440 can be reusable. The first transmission end 431 and the second transmission ends 441 may be pads provided on the first flexible circuit board 430 and the second flexible circuit board 440, respectively. In some embodiments, the first transmission end 431 and the second transmission ends 441 may be connecting contacts provided on the first flexible circuit board 430 and the second flexible circuit board 440, respectively. For example, at least one connecting contact point (which may be referred to as a third connecting contact point) may be provided on the first transmission end 431. At least one connecting contact point (which may be referred to as a fourth connecting contact point) may be provided on each second transmission end 441. In some embodiments, the connecting contact points may be electrical connection points or electrical connection regions on a flexible circuit board.

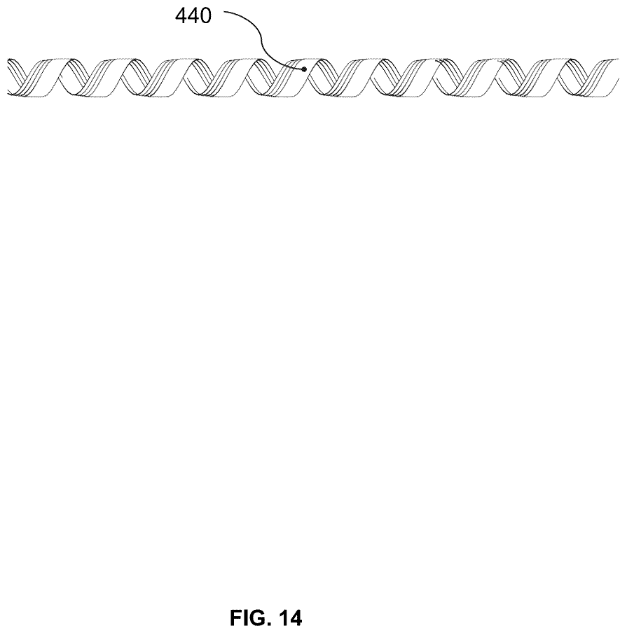

FIG. 14 is a schematic diagram illustrating an exemplary second flexible circuit board 440 according to some other embodiments of the present disclosure.

In some embodiments, the ultrasound probe may include a first portion 450 and a second portion 460 connected in sequence. As shown in FIG. 13, the first connector 300′ (not shown in FIG. 13) and the first flexible circuit board 430 may be disposed within the first portion 450, and the second flexible circuit board 440 may be disposed within the second portion 460. In some embodiments, the second flexible circuit board 440 may be configured in a helical structure as shown in FIG. 14.

In some embodiments, when there are a plurality of the second flexible circuit boards 440 in a catheter, the plurality of the second flexible circuit boards 440 may be stacked sequentially, such that the second flexible circuit board 440 is configured in a spiral structure. When the second flexible circuit board 440 is extended into the second portion configured in a helical structure, the second flexible circuit board 440 configured in a helical structure has a higher space utilization rate and facilitates bending of the second portion.

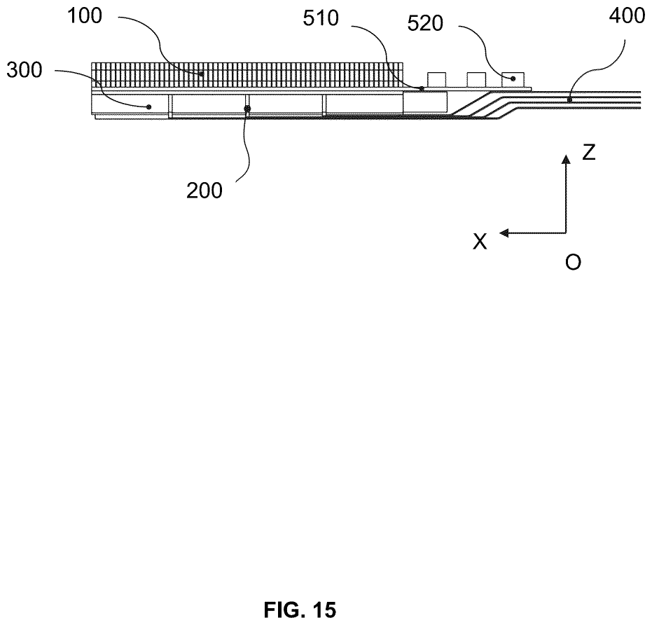

FIG. 15 is a schematic diagram illustrating an internal structure of an exemplary ultrasound probe according to some other embodiments of the present disclosure.

In some embodiments, as shown in FIG. 15, the ultrasound probe may further include an integrated circuit 510 and electronic components 520. The integrated circuit 510 may be provided between the piezoelectric layer 100 and the sound head flexible circuit board 300 and may be electrically connected to the piezoelectric layer 100 and the sound head flexible circuit board 300. The integrated circuit 510 extends outside the piezoelectric layer 100 from one end of the piezoelectric layer 100 along a length direction of the piezoelectric layer (i.e., the OX direction). The electronic components 520 may be disposed on an end of the integrated circuit 510 extending outside of the piezoelectric layer 100.

The integrated circuit 510 and the electronic components 520 may cooperate with a variety of ways to lead out signals in the ultrasound probe. For example, as shown in FIG. 15, the integrated circuit 510 extends outside the piezoelectric layer 100 along an end of the piezoelectric layer 100 proximate the second portion, and the electronic components 520 are provided at an end of the integrated circuit 510 proximate the second portion. The integrated circuit 510 and the electronic components 520 may perform primary processing of signals in the piezoelectric layer 100, and the processed signals are then transmitted to the sound head flexible circuit board 300. The integrated circuit 510 and the electronic components 520 may be directly provided on the sound head flexible circuit board 300, and the sound head flexible circuit board 300 transmits the signals (or processed signals) to the catheter flexible circuit board 400 without the need to set up an adapter board, and the structure is simple. Meanwhile, by replacing the coaxial cables with the catheter flexible circuit board 400, the limitation on the number of coaxial cables due to the inner space of the catheter can be avoided, which addresses the issue of restricted multi-channel transducers. Utilizing the highly integrated and miniaturized advantages of the catheter flexible circuit board 400 can meet the demands for multi-channel transducers.

In some embodiments, the ultrasound probe includes a piezoelectric layer 100, a backing layer 200, a first connector 300′, and a second connector 400′. The backing layer 200 is provided in a laminated arrangement with the piezoelectric layer 100. The first connector 300′ is electrically connected to the piezoelectric layer 100. One end of the second connector 400′ is connected to the first connector 300′, and the other end of the second connector 400′ is connected to a host. The second connector 400′ is configured as at least one flexible circuit board. In some embodiments, the at least one flexible circuit board may be located in a catheter. In some embodiments, the at least one flexible circuit board may be located in at least two catheters in series connection. In some embodiments, as shown in FIG. 9, a flexible circuit board F2 is located in a catheter 470, the flexible circuit board F2 and the catheter 470 may form a catheter portion. In some embodiments, as shown in FIG. 10, two flexible circuit boards F2 are located in a catheter 470, the two flexible circuit boards F2 and the catheter 470 may form a catheter portion. In some embodiments, the at least one flexible circuit board located in the at least one catheter may also be referred to as at least one catheter flexible circuit board. Descriptions regarding the piezoelectric layer 100, the backing layer 200, the first connector 300′, and the second connector 400′ may be found elsewhere in the present disclosure and are not described herein.

### Example 1

As shown in FIG. 4, the ultrasound probe includes a piezoelectric layer 100, a backing layer 200, a sound head flexible circuit board 300, and a catheter flexible circuit board 400. The backing layer 200 is provided in a laminated arrangement with the piezoelectric layer 100. A lamination direction of the piezoelectric layer 100 with the backing layer 200 is a thickness direction of the piezoelectric layer 100 (OZ direction in FIG. 4). A transducer of the ultrasound probe further includes an acoustic lens (not shown in the figure) and an acoustic matching layer (not shown in the figure) stacked sequentially on the piezoelectric layer. The acoustic matching layer is closer to the piezoelectric layer 100 relative to the acoustic lens. The sound head flexible circuit board 300 and the catheter flexible circuit board 400 are configured as flexible printed circuits (FPC).

The sound head flexible circuit board 300 includes a sound head circuit board body 310 and connecting portions 320. The sound head circuit board body 310 is provided between the piezoelectric layer 100 and the backing layer 200 and is electrically connected to the piezoelectric layer 100. The connecting portions 320 are provided at each end of the sound head circuit board body 310 along a width direction (OY direction in FIG. 4), and the connecting portions 320 are sequentially arranged along a length direction (OX direction in FIG. 4) of the piezoelectric layer 100. The connecting portions 320 are provided in pairs, and two connecting portions 320 in the same pair are located at two ends of the sound head circuit board body 310 along the width direction. The two connecting portions 320 are disposed opposite each other along the width direction of the piezoelectric layer 100, which facilitates the connection of the two connecting portions 320 to two lead-out segments 420 disposed on the catheter flexible circuit board 400 simultaneously.

Two adjacent connecting portions 320 disposed at the same end of the sound head circuit board body 310 are disconnected. For example, the two adjacent connecting portions 320 are spaced apart from each other. When folding the connecting portions 320 onto a sidewall of the backing layer 200, a plurality of the connecting portions 320 may be folded one after another, which is easy to operate. The catheter flexible circuit board 400 includes a catheter circuit board body 410 and lead-out segments 420. The lead-out segments 420 are disposed at two ends of the catheter circuit board body 410 along a width direction of the catheter circuit board body 410. The connecting portions 320 are connected one-to-one with the lead-out segments 420 on the catheter flexible circuit board 400, and two adjacent connecting portions 320 are disconnected to facilitate connection of the lead-out segments 420 to the connecting portions 320. As shown in FIG. 4, the connecting portions 320 are connected to the lead-out segments 420 by wire bonding.

The connecting portions 320 include a first connecting segment 321. The first connecting segment 321 extends along the sidewall of the backing layer 200 in a direction away from the piezoelectric layer 100. The catheter flexible circuit board 400 is connected to one end of the first connecting segment 321, and the other end of the first connecting segment 321 is configured to connect to a coupler at a host.

The piezoelectric layer 100 is configured to transmit and receive ultrasonic signals. The piezoelectric layer 100 is provided on the sound head flexible circuit board 300. Signals from the piezoelectric layer 100 may be transmitted to the sound head flexible circuit board 300. The first connecting segment 321 extends outside a laminated region of the backing layer 200 and the piezoelectric layer 100, thereby allowing the signals in the piezoelectric layer 100 to be led out from a sidewall of the piezoelectric layer 100, which is compatible with an arrangement direction of array elements within the piezoelectric layer 100.

The backing layer 200 may have a rectangular structure, and the backing layer 200 has a front side supporting the piezoelectric layer 100, and a backside disposed opposite to the front side, with the front side and the backside connected to each other by the sidewalls. The sound head circuit board body 310 is provided on the front side of the backing layer 200. The first connecting segment 321 is disposed on the sidewall of the backing layer 200.

One end of the catheter flexible circuit board 400 is connected to the connecting portions 320 (first connecting segments 321) and the other end of the catheter flexible circuit board 400 is connected to the coupler at the host for transferring signals in the sound header flexible circuit board 300 to the host, which is configured to process the signals and ultimately display the signal processing results.

By providing the sound head flexible circuit board 300 and the catheter flexible circuit board 400, it is possible to directly transmit the signals in the piezoelectric layer 100 to the host without using an adapter board, an application specific integrated circuit (ASIC), and electronic components that cooperate with the ASIC, reducing a count of the electronic components at a front end of the ultrasound probe, shortening a length of a rigid portion L at the front end, thereby substantially improving the operability of the ultrasound probe. Meanwhile, the coaxial cable is replaced by the catheter flexible circuit board 400, which may avoid the limitations on the number of coaxial cables due to the restricted inner space within the catheter. This embodiment takes advantage of the highly integrated miniaturization of the catheter flexible circuit board 400, the needs of a multi-channel sound head is satisfied.

### Example 2

Different from example 1, as shown in FIG. 5, the connecting portion 320 includes a first connecting segment 321 and a second connecting segment 322 that are connected. The first connecting segment 321 and/or the second connecting segment 322 may include one integral segment. Alternatively, there may be multiple first connecting segments 321 and/or multiple second connecting segments 322 arranged at intervals. When there are multiple second connecting segments 322 arranged at intervals, the second connecting segments 322 may be folded over the backside of the backing layer 200 one after another, which is easy to operate.

The second connecting segment 322 extends along a sidewall of the backing layer 200 to the backside of the backing layer 200 away from the piezoelectric layer 100, and the catheter flexible circuit board 400 is connected to the second connecting segment 322 at the backside of the backing layer 200. In actual production, the sound head circuit board body 310 may first be laid flat on the frontside of the backing layer 200, and then the second connecting segment 322 is folded over the backside of the backing layer 200. One end of the catheter flexible circuit board 400 extends into the backside of the backing layer 200 to connect to the connecting portion 320 (e.g., the second connecting segment 322), and then extends out of the backside of the backing layer 200 to connect to the coupler.

In practice, since the effective length of an array element of the piezoelectric layer 100 determines the size of the entire ultrasound probe along the width direction (OY direction), the effective length cannot be reduced. In this embodiment, the sound head flexible circuit board 300 (or the second connecting segment 322) is folded to the backside of the backing layer 200, and the catheter flexible circuit board 400 is connected to the connecting portion 320 (or the second connecting segment 322) at the backside of the backing layer 200. Compared to leading the catheter flexible circuit board 400 out from two ends of the piezoelectric layer 100 along the width direction (like the lead-out manner in example 1), leading the catheter flexible circuit board 400 out from the backside of the backing layer 200 can reduce the diameter of the entire ultrasound probe and improves the maneuverability of the ultrasound probe. At the same time, the material of the backing layer 200 may be chosen to reduce the thickness of the backing layer 200 and provide more accommodation space for other components (e.g., the catheter flexible circuit board 400).

### Example 3

Unlike example 2, as shown in FIG. 7, the connecting portion 320 includes a first connecting segment 321, a second connecting segment 322 that extends over the backside of the backing layer 200, and a third connecting segment 323 provided at one end of the second connecting segment 322 along the length direction (OX direction), and a catheter flexible circuit board 400 is connected to the third connecting segment 323.

The third connecting segment 323 is provided with pads, and the pads on the third connecting segment 323 are soldered, glued, bonded connected, or integrally molded to pads on the lead-out segment 420. The first connecting segment 321 is wrapped around the outside of a sidewall of the backing layer 200 provided in the length direction, and the second connecting segment 322 is disposed on the backside of the backing layer 200. The third connecting segment 323 is disposed at one end of the second connecting segment 322 along the length direction. In actual use, when the second connecting segment 322 is folded to the backside of the backing layer 200, the third connecting segment 323 is driven to extend outwardly from the backside of the backing layer 200.

The present embodiment provides pads on the third connecting segment 323 (flexible circuit board), and the third connecting segment 323 is connected to the lead-out segment 420 through the pads. Compared with directly providing pads at a rear end of a rigid adapter board and on a printed circuit board (PCB), providing pads on the third connecting segment 323 (flexible circuit board) results in smaller gaps between the pads, and thus allows miniaturization of the ultrasound probe. In addition, since the third connecting segment 323 is of flexible circuit board material, the third connecting segment 323 may be bent toward an end of the backing layer 200 away from the piezoelectric layer 100 (e.g., in an opposite direction of OZ direction as in FIG. 5), which in turn facilitates a reduction in the length of the ultrasound probe (e.g., as the length in the OY direction in FIG. 5).

It should be noted that the foregoing description of the ultrasound probe is intended to be exemplary and illustrative only and does not limit the scope of application of the present disclosure. For a person skilled in the art, various corrections and alterations may be made to the ultrasound probe under the guidance of this application. However, these corrections and changes remain within the scope of the present disclosure.

Some embodiments of the present disclosure also provide an ultrasound imaging device. The ultrasound imaging device includes a host, a coupler, and an ultrasound probe as described above, connected in sequence. One end of the ultrasound probe is connected to the host through the coupler. The ultrasound probe includes a first portion (i.e., a sound head portion) and a second portion (i.e., a catheter portion) connected to each other. The sound head flexible circuit board 300 may be disposed within the sound head portion, and one end of the catheter flexible circuit board 400 away from the sound head flexible circuit board 300 may be extended into the catheter portion and pass through the catheter portion to be connected to the host via the coupler.

In some embodiments, the end of the catheter flexible circuit board 400 away from the sound head flexible circuit board 300 may be provided with a gold finger for docking connection to the coupler. An end of the coupler away from the catheter is connected to the host, which is configured to process the signals and present a processing result.

It should be noted that the foregoing description of the ultrasound imaging device is intended to be exemplary and illustrative only, and does not limit the scope of application of the present disclosure. For a person skilled in the art, various corrections and changes may be made to the ultrasound imaging device under the guidance of the present disclosure. However, these corrections and changes remain within the scope of the present disclosure.

The embodiments of the present disclosure have the following beneficial effects. First, the first connector may be configured as a flexible circuit board, which eliminates the need to set up an adapter board, an application specific integrated circuit (ASIC), or electronic components cooperating with the ASIC. In this way, the ultrasound probe may have a simple structure, a count of components at a front end of the ultrasound probe can be reduced, a length of a rigid portion of the ultrasound probe can be reduced, a volume of the sound head portion can be reduced, and the maneuverability of the ultrasound probe can be improved. Second, the second connector is constructed as a flexible circuit board, by replacing the coaxial cables with the catheter flexible circuit board, the limitation on the number of coaxial cables due to the inner space of the catheter can be avoided, which addresses the issue of restricted multi-channel transducers, thus the needs of a multi-channel sound head is satisfied. Third, the catheter flexible circuit board includes a first flexible circuit board and a second flexible circuit board connected to each other. The first flexible circuit board is provided inside the first portion (i.e., the sound head portion) of the ultrasound probe, and the second flexible circuit board is provided within the second portion (i.e., the catheter portion). When the ultrasound probe is replaced, the first flexible circuit board is replaced at the same time with the ultrasound probe, and the second flexible circuit board may be connected to a first flexible circuit board in the new ultrasound probe to enable reuse of the second flexible circuit board. Fourth, the first flexible circuit board has a first transmission end, and the second flexible circuit board has a plurality of second transmission ends. The plurality of second transmission ends are spaced sequentially along an extension direction of the second flexible circuit board, and the first transmission end is selectively connected to one of the plurality of second transmission ends. When the ultrasound probe is replaced, the second transmission end connected to the first flexible circuit board in the previous ultrasound probe is invalid, and at this time, it is only necessary to connect the replaced ultrasound probe with the other second transmission end to realize the reuse of the second flexible circuit board. It should be noted that the beneficial effects that may be produced by different embodiments are different, and the beneficial effects that may be produced in different embodiments may be any one or a combination of any one or more of the foregoing, or any other beneficial effect that may be obtained.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment,” “one embodiment,” or “an alternative embodiment” in various portions of the present disclosure are not necessarily all referring to the same embodiment. In addition, some features, structures, or characteristics of one or more embodiments in the present disclosure may be properly combined.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. An ultrasound probe, comprising:
a piezoelectric layer (100);
a backing layer (200) provided in a laminated arrangement with the piezoelectric layer (100);
a first connector (300′), wherein
the first connector (300′) is disposed between the piezoelectric layer (100) and the backing layer (200);
the first connector (300′) is electrically connected to the piezoelectric layer (100); and
the first connector (300′) is configured as a flexible circuit board; and
a second connector (400′), wherein one end of the second connector (400′) is connected to the first connector (300′) and the other end is connected to a host.

2. The ultrasound probe of claim 1, wherein:
the first connector (300′) includes a sound head flexible circuit board (300), the sound head flexible circuit board (300) including a sound head circuit board body (310) and one or more connecting portions (320) disposed on at least a portion of a periphery of the sound head circuit board body (310);
the sound head circuit board body (310) is disposed between the piezoelectric layer (100) and the backing layer (200); and
the one or more connecting portions (320) are connected to the second connector (400′).

3. The ultrasound probe of claim 2, wherein:
the one or more connecting portions (320) include a first connecting segment (321), the first connecting segment (321) being disposed on the periphery of the sound head circuit board body (310) and bending away from the piezoelectric layer (100).

4. The ultrasound probe of claim 3, wherein:
the one or more connecting portions (320) further include a second connecting segment (322), the second connecting segment (322) being connected to the first connecting segment (321) and extending over a backside of the backing layer (200) away from the piezoelectric layer (100).

5. The ultrasound probe of claim 4, wherein:
the one or more connecting portions (320) further include a third connecting segment (323) provided at one end of the second connecting segment (322) along a length direction of the second connecting segment (322), and
the second connector (400′) is connected to the third connecting segment (323).

6. The ultrasound probe of claim 2, wherein:
the one or more connecting portions (320) include a plurality of connecting portions (320);
the sound head circuit board body (310) is provided with the plurality of connecting portions (320) at at least one end of the sound head circuit board body (310) along a width direction of the sound head circuit board body (310); and
the plurality of connecting portions (320) are sequentially arranged in at least one row, each row of the at least one row being along a length direction of the piezoelectric layer (100).

7. The ultrasound probe of claim 6, wherein:
two adjacent connecting portions (320) among the plurality of connecting portions (320) at a same end of the sound head circuit board body (310) along the width direction of the sound head circuit board body (310) are disconnected.

8. The ultrasound probe of claim 6, wherein:
the plurality of connecting portions (320) are provided in pairs, and
two connecting portions (320) in a same pair of connecting portions (320) are disposed at two end of the sound head circuit board body (310) along the width direction of the sound head circuit board body (310).

9. The ultrasound probe of claim 2, further comprising an integrated circuit (510) and one or more electronic components (520), wherein:
the integrated circuit (510) is provided between the piezoelectric layer (100) and the sound head flexible circuit board (300) and is electrically connected to the piezoelectric layer (100) and the sound head flexible circuit board (300),
the integrated circuit (510) extends outside the piezoelectric layer (100) from one end of the piezoelectric layer (100) along a length direction of the piezoelectric layer (100), and
the one or more electronic components (520) are disposed on an end of the integrated circuit (510) extending outside of the piezoelectric layer (100).

10. The ultrasound probe of claim 1, wherein:
the second connector (400′) includes at least one catheter flexible circuit board (400); and
the at least one catheter flexible circuit board (400) is electrically connected to the first connector (300′).

11. The ultrasound probe of claim 10, wherein:
the first connector (300′) includes a sound head flexible circuit board (300), the sound head flexible circuit board (300) including a sound head circuit board body (310) and one or more connecting portions (320) disposed on at least a portion of a periphery of the sound head circuit board body (310),
the sound head circuit board body (310) is disposed between the piezoelectric layer (100) and the backing layer (200),
the one or more connecting portions (320) are connected to the at least one catheter flexible circuit board (400), and
for each of the at least one catheter flexible circuit board (400), the catheter flexible circuit board (400) includes a catheter circuit board body (410) and one or more lead-out segments (420), the one or more lead-out segments (420) being located at at least one end of the catheter circuit board body (410) in a width direction of the catheter circuit board body (410), and the one or more lead-out segments (420) being electrically connected to the one or more connecting portions (320).

12. The ultrasound probe of claim 11, wherein:
the at least one catheter flexible circuit board (400) includes a plurality of catheter flexible circuit boards (400),
each of a plurality of catheter circuit board bodies (410) of the plurality of catheter flexible circuit boards (400) is provided with the one or more lead-out segments (420) at two ends of the catheter circuit board body (410) along the width direction of the catheter circuit board body (410), and
two lead-out segments (420) among the one or more lead-out segments (420) on a same catheter flexible circuit board (400) are connected to a pair of connecting portions (320) among the one or more connecting portions (320) in a one-to-one correspondence, respectively.

13. The ultrasound probe of claim 11, wherein:
the at least one catheter flexible circuit board (400) includes a plurality of catheter flexible circuit boards (400),
each of a plurality of catheter circuit board bodies (410) of the plurality of catheter flexible circuit boards (400) is provided with the one or more lead-out segments (420) at one end of the catheter circuit board body (410) along the width direction of the catheter circuit board body (410), and
a pair of connecting portions (320) among the one or more connecting portions (320) are connected to two lead-out segments (420) respectively disposed on two catheter flexible circuit boards (400) among the plurality of catheter flexible circuit boards (400).

14. The ultrasound probe of claim 11, wherein the one or more connecting portions (320) are welded, bonded, bound together or integrally molded with the one or more lead-out segments (420).

15. The ultrasound probe of claim 10, wherein:
for each of the at least one catheter flexible circuit board (400), the catheter flexible circuit board (400) includes a first flexible circuit board (430) and a second flexible circuit board (440) connected to each other,
an end of the first flexible circuit board (430) away from the second flexible circuit board (440) is connected to the first connector (300′), and
an end of the second flexible circuit board (440) away from the first flexible circuit board (430) is connected to the host.

16. The ultrasound probe of claim 15, wherein:
the first flexible circuit board (430) includes a first transmission end (431),
the second flexible circuit board (440) includes a plurality of second transmission ends (441),
the plurality of second transmission ends (441) are spaced sequentially along an extension direction of the second flexible circuit board (440), and
the first transmission end (431) is selectively connected to one of the plurality of second transmission ends (441).

17. The ultrasound probe of claim 15, wherein:
the ultrasound probe includes a first portion and a second portion connected in sequence,
the first connector (300′) and the first flexible circuit board (430) are located within the first portion,
the second flexible circuit board (440) is located within the second portion, and
the second flexible circuit board (440) is configured in a helical structure.

18. The ultrasound probe of claim 10, wherein:
the at least one catheter flexible circuit board (400) includes at least one flexible circuit board in a catheter.

19. An ultrasound imaging device including a host, a coupler, and an ultrasound probe connected in sequence, one end of the ultrasound probe being connected to the host through the coupler, wherein the ultrasound probe includes:
a piezoelectric layer (100);
a backing layer (200) provided in a laminated arrangement with the piezoelectric layer (100);
a first connector (300′), wherein
the first connector (300′) is disposed between the piezoelectric layer (100) and the backing layer (200);
the first connector (300′) is electrically connected to the piezoelectric layer (100); and
the first connector (300′) is configured as a flexible circuit board; and
a second connector (400′), wherein one end of the second connector (400′) is connected to the first connector (300′) and the other end is connected to a host.

20. An ultrasound probe, comprising:
a piezoelectric layer (100);
a backing layer (200) provided in a laminated arrangement with the piezoelectric layer (100);
a first connector (300′) electrically connected to the piezoelectric layer (100); and
a second connector (400′), wherein
one end of the second connector (400′) is connected to the first connector (300′) and the other end is connected to a host;
the second connector (400′) is configured as at least one flexible circuit board; and
the at least one flexible circuit board is in a catheter.
